(19) United States
(12) Patent Application Publication
(10) Pub. No.: US 2024/0350221 A1
(43) Pub. Date: Oct. 24, 2024

(54) TAMPERPROOF SURGICAL BACK TABLE DRAPE

(71) Applicant: Creative Surgical Solutions, LLC, Vail, CO (US)

(72) Inventors: James Bemman, Holly, MI (US); Samba Toure, Grand Blanc, MI (US); Nicholas Sievers, Clarkston, MI (US); Phillip Reed Brown, McHenry, IL (US); Brian L. Wilt, Appleton, WI (US); Evelina Leece, Chicago, IL (US); Wojciech Porcelk, Davison, MI (US); Donald Corenman, Edwards, CO (US); Eric Strauch, Vail, CO (US)

(21) Appl. No.: 18/763,679

(22) Filed: Jul. 3, 2024

Related U.S. Application Data
(63) Continuation of application No. 16/499,399, filed on Sep. 30, 2019, now abandoned.
(60) Provisional application No. 62/480,021, filed on Mar. 31, 2017.

(51) Int. Cl. A61B 46/10; A61B 17/00; A61B 46/20; A61B 50/15; A61B 90/00

(52) U.S. Cl. CPC A61B 46/10; A61B 50/15; A61B 2017/00876; A61B 2046/205; A61B 2090/0807

(57) ABSTRACT

Systems and methods for using a sterile drape with a surgical table are described. The system includes a sterile drape and at least one securement device that secure the sterile drape to the surgical table. The securement device may be an adhesive sticker having a perforation. Because of the perforation, the sticker is prone to break under small levels of force exerted on the sticker. In this way, a user can visually determine whether potential breaches of sterility have occurred when the sticker has broken. Additionally, the system may include magnets, weights, poly-ties, rubber bands, hooks, clips, buckles, adhesive tabs, straps, tape, and the like to secure the sterile drape to the surgical table. In this way, the surgical table can be prepared, the sterile drape can be installed, and the drape can be secured to the table to prevent breaches of sterility.

### CROSS-REFERENCE TO RELATED APPLICATION

This application is a continuation of U.S. patent application Ser. No. 16/499,399, filed Sep. 30, 2019, which is a national stage application under 35 U.S.C. 371 and claims the benefit of PCT Application No. PCT/US2018/025330 having an international filing date of 30 Mar. 2018, which designated the United States, which PCT application claimed the benefit of U.S. App. No. 62/480,021 filed on Mar. 31, 2017, the disclosures of each of which are incorporated by reference herein.

### BACKGROUND OF THE INVENTION

### 1. Field of the Invention

The present invention relates in general to the field of medical procedures. More particularly, the present invention relates to drapes used during medical procedures to improve sterile conditions. Specifically, a preferred embodiment of the present invention relates to a drape that is used to maintain sterility about a surgical back table holding medical instruments prior to a medical procedure.

### 2. Discussion of the Related Art

In the operating room, a back table is an area where there is major risk of contamination. Traditionally, the various pieces of medical equipment that will be utilized during the medical procedure are placed onto the back table prior to the surgery. More specifically, a sterile cover is applied to the back table, and then the medical equipment is placed upon the cover. Because the bulk of these pieces of medical equipment will come into direct contact with a patient, it is imperative that all of the equipment remains sterile prior to the surgical procedure.

As a result, contamination about a back table is a major problem during surgical procedures. Back tables can be contaminated in as little as 30 minutes when exposed to air in the operating room. In fact, The Journal of Bone and Joint Surgery has found that approximately 4% of back tables are contaminated after 30 minutes, 15% of back tables are contaminated after an hour, 22% of back tables are contaminated after two hours, and 30% are contaminated after four hours. Obviously, these contaminations can present significant health concerns to patients and significant costs to the hospital or medical facility.

As a result, current standard practice is that once the medical equipment is placed onto the back table, the back table must not be “left unobserved.” See 2017 AORN Guidelines for Perioperative Practice. Nurses usually refer to this as keeping the back table attended. Further, this has commonly been interpreted to mean that a member of the medical staff must stay with the back table and equipment lying thereupon to ensure that sterility about the table is maintained. Obviously, this results in additional costs to the hospital or medical facility in terms of human resources. Additionally, by requiring constant observance of the back table, it is impractical to prepare the back table, including installing the cover and organizing the medical equipment, far in advance of the surgery.

It should also be noted that even when a back table is attended, this does not protect the back table from contamination in the air, such as by dust, bacteria, air circulation, and the like.

U.S. Pat. No. 8,726,907 and Application Publication Nos. 2014/0251346 and 2015/0374442, all of which are incorporated herein by reference in their entirety, have introduced a number of different drapes used throughout the medical field to help avoid potential breaches in sterility. While many of the drapes shown in this patent and applications could be used to help maintain sterility about the back table, there is still a need to provide a drape that provides a sufficient alternative to having the back table under constant observation as suggested by the 2017 AORN Guidelines.

What is needed therefore is a drape system that can be used with a surgical back table to ensure that sterility is maintained about the back table and the instruments resting thereon without constant observation by a member of the medical staff. What is also needed is a drape system that provides a visual indicator that allows medical practitioners to quickly and easily determine whether a prepared back table, including a back table drape has been tampered with, which could result in a loss of sterility. What is further needed is a drape system that allows the back table to be assembled and sterility to be maintained prior to the surgical procedure. What is also needed is a back table drape that is transparent, which allows a user to visually inspect the contents of the back table.

### SUMMARY AND OBJECTS OF THE INVENTION

By way of summary, the present invention is directed to a drape system. A primary object of the invention is to provide a drape system that is used with a surgical table including a sterile drape and at least one securement device. The surgical table is supported by a plurality of legs with an underside and a top surface with a perimeter. The top surface is used to support a plurality of surgical instruments.

Another object of the invention is to provide a sterile drape that may have a first edge, a second edge, a first end, a second end, a bottom side, a perforation, and an overlapping portion. In one embodiment, the perforation extends from the first end to the second end between the first side and the second side. The overlapping portion is located above the perforations.

Additionally, the sterile drape may include an area defined by a predetermined length and a predetermined width. The drape may also have a selectively separable portion extending a substantial portion of at least one of the predetermined length and the predetermined width. The selectively separable portion further includes a first peripheral edge and a second peripheral edge. These peripheral edges are initially in a sealed position but can be unsealed by applying opposing forces to the peripheral edges. Additionally, the selectively separable portion includes a first overlaying section which overlays the selectively separable portion when the sterile drape is in a sealed position.

Yet another object of the invention is to provide a securement device that is used to secure the sterile drape to the surgical table. Preferably, the securement device is configured to indicate when the sterile drape has been moved from the surgical table. This allows a user to ensure that sterility is maintained about the top surface and the plurality of surgical instruments.

For instance, in yet another aspect of this invention, the securement device is an adhesive sticker with a first side, a second side opposite the first side, a third side extending between the first side and the second side, and a fourth side extending between the first side and the second side and located opposite the third side. The adhesive sticker also has a perforation that extends from the first side to the second side. The perforation is configured to easily break when the adhesive sticker is pulled apart. In one embodiment, the third side of the adhesive sticker is attached to one of the first edge, the second edge, the first end, or the second end of the sterile drape and the fourth side is attached to one of the underside or one of the plurality of legs.

In another aspect of the invention, the system can additionally include one of a magnet and a weight that is rested on top of the sterile drape. The magnet may form a magnetic connection with the table. Both a magnet and a weight help to keep the sterile drape secured to the table.

In yet another aspect of the invention, the sterile drape may have at least one poly-tie. Each poly-tie has a first end permanently attached to the sterile drape, a second end with an adhesive, and a release liner covering the adhesive. When the poly-tie is used, the release liner can be removed to expose the adhesive and the second end is pulled away from the first end. The adhesive is then secured to the sterile drape to secure the sterile drape to the table. Once the second end is secured to the sterile drape, a third side of the adhesive sticker is attached to the second end of the poly-tie and the fourth side of the adhesive sticker is attached to the sterile drape. In this way, if the second end of the poly-tie is detached from the sterile drape, the adhesive sticker will break about the perforation to provide visual evidence that sterility about the table may have been compromised. Alternatively, the system can also include a buckle through which the poly-tie may be secured. The third side of the adhesive sticker is then attached to the poly-tie and the fourth side of the adhesive sticker is attached to the sterile drape.

In another aspect of the invention, the system may include a plurality of rubber bands and a plurality of hooks that are used to engage adjacent rubber bands. These rubber bands are wrapped around the perimeter, and a third side of the adhesive sticker is attached to one of the hooks, and the fourth side of the adhesive sticker is attached to the sterile drape. In this way, if the hook is disengaged from the rubber bands, the adhesive sticker will break about the perforation to provide visual evidence that sterility about the table may have been compromised.

In another aspect of the invention, the system may include a plurality of clips that releasably attach to the plurality of legs. In doing so, the clips secure the sterile drape to the plurality of legs. The third side of the adhesive sticker is then attached to the clip, and the fourth side of the adhesive sticker is attached to the sterile drape. In this way, if the clip is detached from the leg, the adhesive sticker will break about the perforation to provide visual evidence that sterility about the table may have been compromised.

In another aspect of the invention, the securement device includes a plurality of adhesive tabs located on the bottom side of the sterile drape and a plurality of release liners releasably attached to the adhesive tabs. When the sterile drape is ready to be used, the release liners are removed to expose the adhesive tabs, and the tabs are anchored to the top surface of the table. In the event that any of the adhesive tabs are not anchored to the top surface, it will be evident that sterility about the table may have been compromised. Alternatively, the adhesive tabs can be anchored to the perimeter. Additionally, the sterile drape may have a perforation that extends around the perimeter.

In yet another aspect of the invention, the securement device includes at least one strap with a first end, a second end, and a body located therebetween. The first end is attached to one of the plurality of legs, the body secures the sterile drape to the table, and the second end is attached to another one of the plurality of legs. The third side of the adhesive sticker is attached to one of the first end and the second end and the fourth side of the adhesive sticker is attached to one of the plurality of legs. In this way, if the end is detached from the leg, the adhesive sticker will break about the perforation to provide visual evidence that sterility about the table may have been compromised. Additionally, the strap could be reusable.

In another aspect of the invention, the securement device includes at least one strip of tape with a first end, a second end, and a body therebetween. The first end of the tape is attached to one of the plurality of legs, the body is secured to the sterile drape, and the second end of the tape is attached to another of the plurality of legs.

In accordance with another object of the invention, a method of maintaining sterility about a back table is provided. This method can include placing a sterile cover on a top surface of the back table. After the cover is placed, a plurality of sterile surgical instruments may be placed on the sterile cover. Next, a sterile drape is placed on top of the sterile surgical instruments. After the drape has been placed, a securement device is used to secure the sterile drape to the surgical table. The securement device is configured to indicate when the drape has been removed from the surgical table to ensure sterility is maintained about the top surface and the plurality of surgical instruments.

In accordance with another aspect of the invention, the method may include additional steps. For instance, an adhesive sticker that is the securement device can be provided. The adhesive sticker may include a first side, a second side, a third side, and a fourth side, with a perforation located between the third side and the fourth side. The third side of the adhesive sticker is then attached to one of a plurality of legs supporting the table, and the fourth side of the adhesive sticker is attached to the sterile drape. Once it is time to perform the surgical procedure, the adhesive sticker may be broken in half about the perforation by pulling the third side of the adhesive sticker away from the fourth side of the adhesive sticker.

These and other aspects and objects of the present invention will be better appreciated and understood when considered in conjunction with the following description and the accompanying drawings. It should be understood, however, that the following description, while indicating preferred embodiments of the present invention, is given by way of illustration and not of limitation. Many changes and modifications may be made within the scope of the present invention without departing from the spirit thereof, and the invention includes all such modifications.

### BRIEF DESCRIPTION OF THE DRAWINGS

A clear conception of the advantages and features constituting the present invention, and of the construction and operation of typical mechanisms provided with the present invention, will become more readily apparent by referring to the exemplary, and therefore non-limiting, embodiments illustrated in the drawings accompanying and forming a part of this specification, wherein like reference numerals designate the same elements in the several views, and in which:

FIG. 1 illustrates an inventive tamperproof surgical back table drape;

FIG. 2 illustrates the surgical back table drape showing a double Z flap orientation in a sealed position;

FIG. 3 illustrates the surgical back table drape of FIG. 2 showing the double Z flap orientation being pulled apart toward an unsealed position;

FIG. 4 illustrates the surgical back table drape of FIGS. 2 and 3 where the two sides of the surgical back table drape separate at a perforation;

FIG. 5 illustrates the surgical back table drape showing a single Z flap as it is moved from a sealed positon to an unsealed position;

FIG. 6 illustrates the surgical back table drape showing a securement device that is an adhesive sticker with a perforation;

FIG. 7 illustrates the surgical back table drape with a magnet that forms a magnetic connection with either the surgical back table or a magnet affixed to the surgical back table, such that the surgical back table drape is secured to the surgical back table;

FIG. 8 illustrates the surgical back table drape with a weight that secures the surgical back table drape to the surgical back table;

FIG. 9 illustrates the surgical back table drape with a poly-tie that secures the surgical back table drape to the surgical back table around the corner of the surgical back table;

FIG. 10 illustrates the surgical back table drape with a poly-tie that secures the surgical back table drape to the surgical back table beneath an underside of the surgical back table, where a release layer is being removed from a second end of the poly-tie to expose an adhesive;

FIG. 11 illustrates the surgical back table drape where the adhesive is being secured to the back table drape;

FIG. 12 illustrates the surgical back table drape where the adhesive has been secured to the back table drape;

FIG. 13 illustrates the surgical back table drape where an adhesive sticker is being applied to the second end of the poly-tie and to the surgical back table drape;

FIG. 14 illustrates the surgical back table drape where an adhesive sticker is applied to the second end of the poly-tie and to the surgical back table drape;

FIG. 15 illustrates the surgical back table drape where the adhesive sticker has been broken about a perforation, evidencing that sterility about the surgical back table drape may have been compromised;

FIG. 16 illustrates the surgical back table drape where a plurality of rubber bands and a plurality of hooks are included with the surgical back table drape;

FIG. 17 illustrates the plurality of rubber bands and the plurality of hooks that are included with the surgical back table drape in greater detail;

FIG. 18 illustrates another view of the plurality of rubber bands and the plurality of hooks that are included with the surgical back table drape in greater detail;

FIG. 19 illustrates the plurality of rubber bands and the plurality of hooks once they are installed to secure the surgical back table drape to the surgical back table;

FIG. 20 illustrates another view of the plurality of rubber bands and the plurality of hooks once they are installed to secure the surgical back table drape to the surgical back table;

FIG. 21 illustrates yet another view of the plurality of rubber bands and the plurality of hooks once they are installed to secure the surgical back table drape to the surgical back table;

FIG. 22 illustrates the surgical back table drape where an adhesive sticker is applied to one of the hooks and to the surgical back table drape;

FIG. 23 illustrates a clip that is used to secure the surgical back table drape to one of the legs of the surgical back table;

FIG. 24 illustrates the clip of FIG. 23 after it is releasably connected to one of the legs of the surgical back table;

FIG. 25 illustrates a strap used to secure the surgical back table drape to the surgical back table;

FIG. 26 illustrates another view of the strap used to secure the surgical back table drape to the surgical back table;

FIG. 27 illustrates a drape with a plurality of adhesive tabs located on the underside of the drape;

FIG. 28 illustrates the surgical back table drape where excess plastic is folded and then secured into place using an adhesive tab;

FIG. 29 illustrates the surgical back table drape with at least one fold that can be gathered and secured into place using an adhesive tab;

FIG. 30 illustrates the surgical back table drape with at least one slit where material on either side of the slit is gathered and secured into place using an adhesive tab;

FIG. 31 illustrates the surgical back table drape where pull tabs may be mounted to opposite corners of the drape that can be pulled in opposite direction to gather excess material and secure it into place;

FIG. 32 illustrates the surgical back table drape where pull tabs are mounted at each corner of the drape that can be pulled to gather excess material and secure it into place;

FIG. 33 illustrates the surgical back table drape where an additional dome bag is placed on top of the surgical back table drape to secure any excess material into place; and

FIG. 34 illustrates the surgical back table drape having a box-shaped cover.

In describing the preferred embodiment of the invention which is illustrated in the drawings, specific terminology will be resorted to for the sake of clarity. However, it is not intended that the invention be limited to the specific terms so selected and it is to be understood that each specific term includes all technical equivalents which operate in a similar manner to accomplish a similar purpose. For example, the word connected, attached, or terms similar thereto are often used. They are not limited to direct connection but include connection through other elements where such connection is recognized as being equivalent by those skilled in the art.

### DESCRIPTION OF PREFERRED EMBODIMENTS

The present invention and the various features and advantageous details thereof are explained more fully with reference to the non-limiting embodiments described in detail in the following description.

### 1. System Overview

The current invention is directed to a sterile drape for use with a surgical table that has a tamperproof feature that provides a visual indicator to help a user easily determine if sterility has been compromised about the table. The drape is a sterile cover that helps reduce the risk of contamination from airborne micro-organisms. The drape can be used over the top of a surgical back table that is positioned inside an operating room before an upcoming clinical procedure. These tables traditionally remain in a static condition (i.e. non-mobile table) when they are prepared. A surgical technician first places a sterile back table cover over the top surface of the surgical back table. Next, the surgical technician places sterile surgical tools and instruments on top of the cover to prepare for an upcoming clinical procedure.

Current AORN Guidelines for perioperative practice require that the sterile field is not subject to unrecognized contamination by personnel, vectors (e.g. insects), or breaks in the sterile technique, if left unobserved. The language relating to being “unobserved” has been interpreted to mean that once the back table has been prepared, a sterile field should not be left unattended. This means that an employee of the hospital must remain by the back table to ensure sterility is maintained. However, by introducing a tamperproof securement device to the drape, sterility can be maintained about the back table without having to rely on an employee to constantly observe the back table. In order for this to occur, airflow about the perimeter edges of the drape must not lift the edge of the drape.

A number of different securement devices could be used. For instance, the sterile drape could have built-in weights along the edges to prevent airflow from lifting the perimeter edges. Additionally, tamperproof stickers are applied to the drape and to the legs of the table or the underside of the table. In the event that the sticker is torn, it will be clear to individuals observing the table that sterility may have been breached.

Additionally, magnets could be used that form a magnetic connection with the table. Alternatively, in the event that the table is not made of a magnetic material, an adhesive-backed magnetic strip can be installed to the table. A magnet could then be placed on top of the magnetic strip in order to form a magnetic connection. Again, tamperproof stickers are applied to the drape and to the legs of the table or the underside of the table. In the event that the sticker is torn, it will be clear to individuals observing the table that sterility may have been breached.

Further still, poly-ties could be used with the sterile drape. For instance, poly-ties could be located at each of the four corners of the sterile drape. Each poly-tie is fastened to the drape, and tamperproof stickers are applied to the poly-tie and to the drape. Alternatively, a buckle or multiple buckles could be provided that the poly-tie can be inserted into. Also, a single long poly-tie can be wrapped around the perimeter of the table. Additionally, where a single long poly-tie is used, loops can be located around the perimeter to help guide where the poly-tie should be. Once installed, a tamperproof sticker is applied to the poly-tie and the drape or a portion of the table. If the sticker is torn, it will be clear that sterility may have been breached.

Alternatively still, a plurality of rubber bands and hooks could be used in combination to wrap around the table. Tamperproof stickers could then be fastened to the hooks and the drape or a portion of the table. If the sticker is torn, it will be clear that sterility may have been breached.

Also, clips may be used that are configured to snap over the legs of the back table. In use, the sterile drape would first be placed about the table. The sides of the drape are pulled downwardly and placed against each leg of the table before the clips are then snapped to secure the drape between the clip and the leg. Tamperproof stickers could then be fastened on one side to the clip and on the other side to one of the drape, the legs, or the underside of the table. Alternatively, the clips could be configured to be breakable when removed from the legs. This would allow a user to quickly and easily identify a potential breach of sterility where the clip is broken or the sticker is torn.

Further still, a plurality of adhesive tabs may be included on the underside of the drape, each of which is covered by a release liner. When unfolding the cover, the release liners are removed to expose the adhesive tabs. The adhesive tabs are then pressed against the table in order to anchor the drape to the table top. Alternatively, the adhesive tabs may be pressed against the perimeter of the table. A user can then visually inspect the adhesive tabs to see if any have been disengaged, which is evidence of a potential breach in sterility. Similarly, perforations can be utilized around the tabs, where the perforations are inspected for any evidence of tampering, which would result in potential breaches of sterility. The adhesive tabs could also be color coded with a top portion being a first color and a bottom portion being a second color. This would help to allow a user to quickly identify whether or not the tab has been broken.

Alternatively, a strap or straps could be used to secure the drape about the table. For instance, the strap could be situated such that a first end is secured to one of the table legs, and a second end is secured to the other of the table legs. For instance, the strap could form an “X” pattern or a “Z” pattern. The strap could be reusable and it could also be color coded. Once the strap is in place, tamperproof stickers could be applied to the strap and the legs or the table.

Additionally, a large zip-lock bag could be used that contains the sterile instruments. The bag could be configured to allow a user to visually understand whether the bag has remained sealed, for instance, using a bag with color changing technology that would result in a change of color once the bag is opened. Similarly, the bag could be vacuum-sealed such that it will be easy for a user to visually confirm whether the bag has remained sealed based on whether the bag is still vacuumed. In the event that the bag is not still vacuumed, it will be apparent that the seal has been broken, and that sterility might have been compromised. Also, a sterilized gas could similarly be applied to the drape and the surrounding components at any time to ensure sterility about the table and associated components.

It should also be noted that the drape or securement device could be moved or removed and later reapplied. By reapplying securement devices or applying new securement devices after the drape has either been moved or removed, risk of airflow about the table can be minimized, which in turn helps to maintain sterility.

Also, any of the drapes described above could also feature a time stamp. This could include pertinent information, including who prepared the back table, when it was prepared, what operation the table was prepared for, and identifying information about the patient. Alternatively still, an RF/ID tag could be installed onto the cover or drape, which would allow a smartphone, tablet, scanner, etc. to scan the label and capture date and time information once the drape is placed.

Any of the drapes described above could include a single or multiple sterile sheets of plastic. Alternatively, any of the drapes described above could include a single or multiple sterile sheets of patient drape material. Also, any of the drapes described above could include a single or multiple sterile sheets of fabric. Similarly, any combination of the above-discussed drapes could be used in combination with a perforation line. Additionally, a sterile dome bag and a sterile cinch bag could be used. Also, tamper evident tapes and labels could be used with any of the disclosed drape systems.

### 2. Detailed Description of Preferred Embodiments

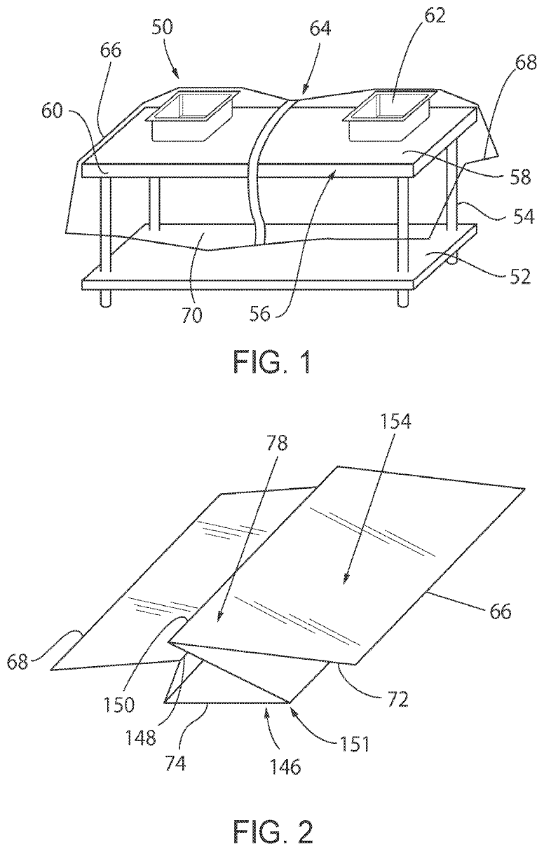

Turning to the figures, a drape system 50 with a sterile drape 64 and a securement device 80 for use with a table 52 is shown generally in FIG. 1. As shown, the table 52 is a back table for use during surgical procedures. The table 52 has four legs 54 that support a table top with a top surface 58, an underside 56, and a perimeter 60. The top surface 58 is configured to support a plurality of surgical instruments 62, which obviously could vary depending on the specific surgical procedure that is set to occur. A back table cover 53 may be rested on the top surface 58 with the instruments 62 resting on the cover 53.

Specific embodiments of the present invention will now be further described by the following, non-limiting examples which will serve to illustrate various features of significance. The examples are intended merely to facilitate an understanding of ways in which the present invention may be practiced and to further enable those of skill in the art to practice the present invention. Accordingly, the examples should not be construed as limiting the scope of the present invention.

The sterile drape 64 includes a first edge 66, a second edge 68, a first end 70, and a second end 72. The first edge 66 and the second edge 68 form a predetermined length. The first end 70 and second end 72 form a predetermined width. The predetermined length and the predetermined width form an area. A perforation 151 is found in a bottom side 74 of the drape 64, with an overlapping portion 78 located above the perforation 151 at a selectively separable portion 146 covered by an overlaying section 154. Additionally, it should be noted that the sterile drape 64 is transparent. This is helpful because the back table 52 and its contents can remain visible by medical practitioners.

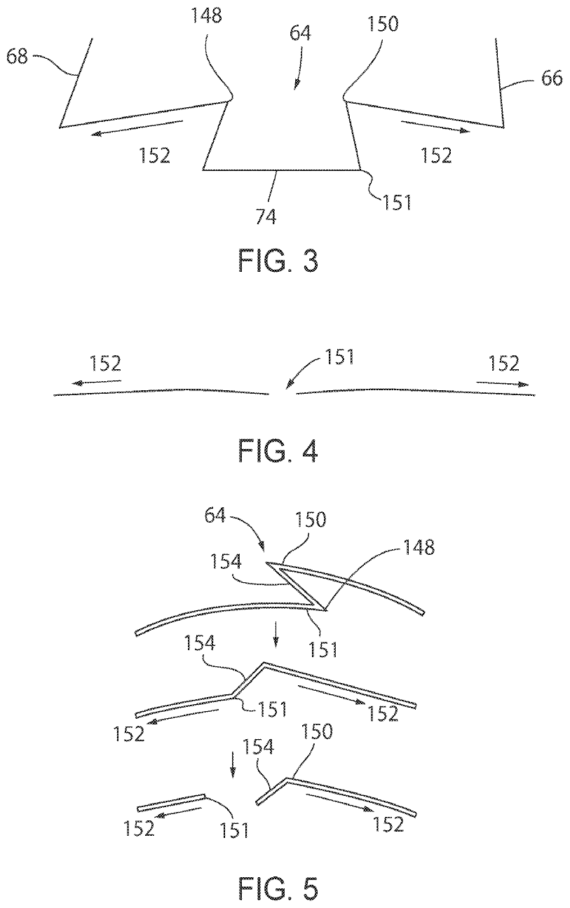

The overlapping portion 78 may form a “double Z” flap orientation, with a first peripheral edge 148 and a second peripheral edge 150, as shown in FIGS. 2-4. FIG. 2 shows the drape 64 in an initial sealed position 153. Alternatively, the overlapping portion 78 may form a “single Z” flap orientation with a first peripheral edge 148 and a second peripheral edge 150, as shown in FIG. 5. The perforation 151 and the rest of the drape 64 overlie the sterile field found about the table 52. When a user is ready to remove the drape 64, the user applies a pulling force 152 to pull apart the first edge 66 and the second edge 68, and the drape portions separate along the perforation 151, as seen in FIGS. 3-5. The two halves of the drape may thus be pulled or allowed to fall over opposing sides of the sterile field without compromising the sterility of the sterile field about the table 52. The “double Z” configuration of the drape 64 shown in FIGS. 2-4 and the “single Z” configuration of the drape 64 shown in FIG. 5 maintain a sterile surface adjacent the perforation 151. Although a perforated separable drape 64 is described in these embodiments, it is expressly understood that other means of providing a predetermined separable portion 146 of the drape 64 are included and considered within the scope of the invention. For example, a surface with scoring along the predetermined separable portion 146 may be provided. As another example, the drape 64 may be comprised of a different material along the predetermined separable portion 146, wherein the different material comprises a characteristic which allows it to be torn or separated more easily than the material of the remainder of the drape 64.

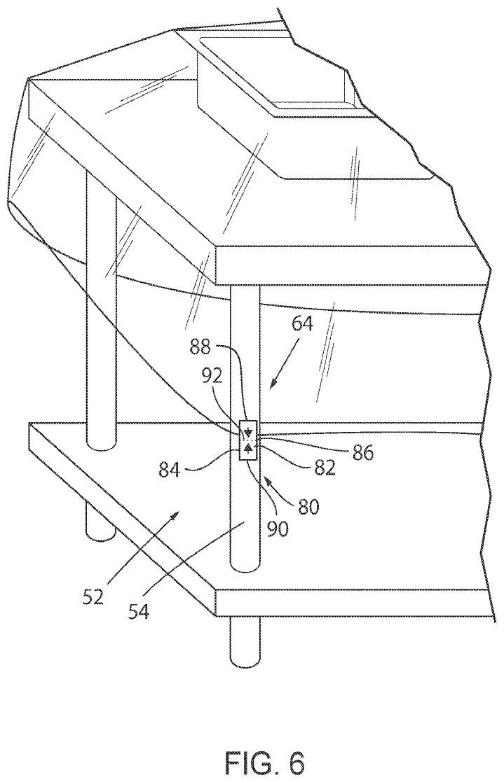

Various securement devices 80 will now be described. Turning now to FIG. 6, the securement device 80 may be an adhesive sticker 82. As shown, the adhesive sticker 82 has a first side 84, a second side 86, a third side 88, and a fourth side 90. A perforation 92 is formed in the adhesive sticker 82 from the first side 84 to the second side 86 and between the third side 88 and the fourth side 90. In a number of the embodiments shown in the figures, the adhesive sticker 82 is secured to an edge or end of the sterile drape 64 and the one of the legs 54 or the underside 56 of the table 52. In this way, if the drape 64 is moved away from the legs 54 or the underside 56 of the table 52, the perforation 92 will break. As a result, a user can visually inspect the sticker 82 to determine whether there is risk that sterility about the table 52 has been breached. If the sticker 82 is broken about the perforation 92, the user can remove the existing drape 64, instruments 62, and cover 53 and rebegin the table 52 draping process to ensure that sterility is maintained. The securement device 80 may include text, symbols, or other indicia to instruct a user as to how the device should be used or broken.

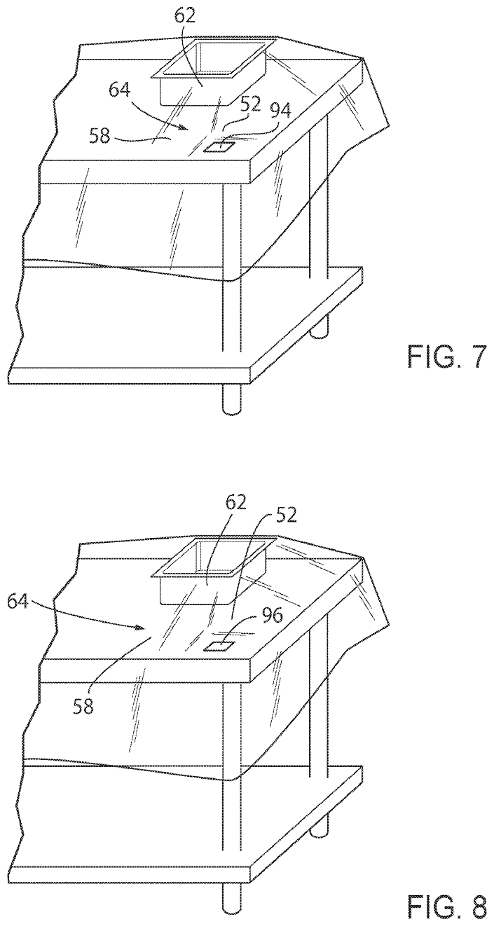

In addition to using the adhesive sticker 82 to help confirm that sterility is maintained about the drape system 50, additional components can be used to keep the drape 64 secure to the table about the perimeter 60. For instance, turning to FIG. 7, a magnet 94 is provided. The magnet 94 forms a magnetic connection with the top surface 58 of the table 52 to prevent the drape 64 from coming up around the perimeter 60. Alternatively, where the table 52 is not made of a magnetic surface, a second magnet (not shown) with an adhesive back (not shown) can first be installed on the top surface 58 of the table 52 or to the cover 53. The drape 64 is then placed on top of the top surface 58, and the magnet 94 is placed on top of the drape 64 to engage the second magnet. In this way, movement of the drape 64 can be minimized to reduce the risk of contamination from airborne micro-organisms. Once the drape 64 is placed and the magnet 94 is applied, an adhesive sticker 82 can be applied as described above.

Somewhat similarly, turning to FIG. 8, a weight 96 or plurality of weights can be used to prevent the drape 64 from coming up around the perimeter 60. The weight 96 may be rested on top of the drape 64 after the drape 64 is placed on the table 52, or the weight 96 could be built into, or manufactured with the drape 64. Preferably, the weights 96 are located around the edge of the perimeter 60 to ensure the drape 64 remains in place. Once the drape 64 is placed and the weight 96 is applied, an adhesive sticker 82 can be applied as described above.

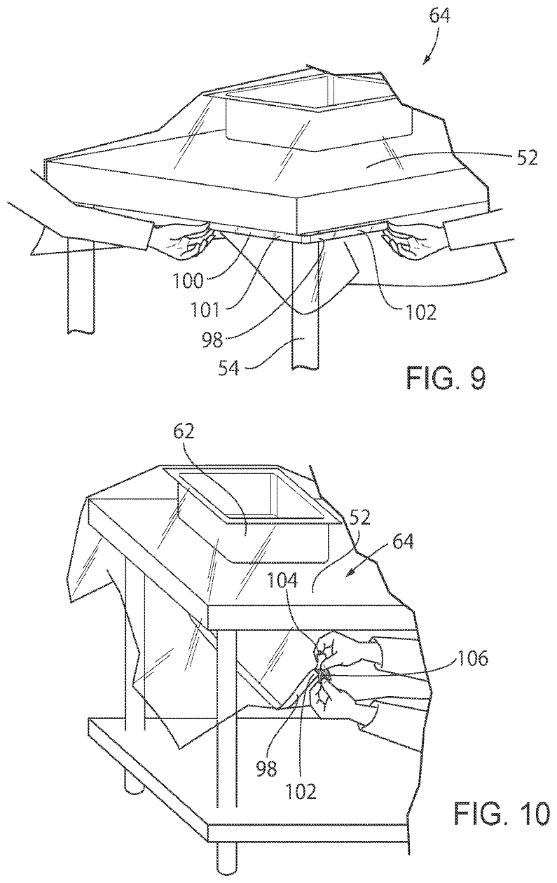

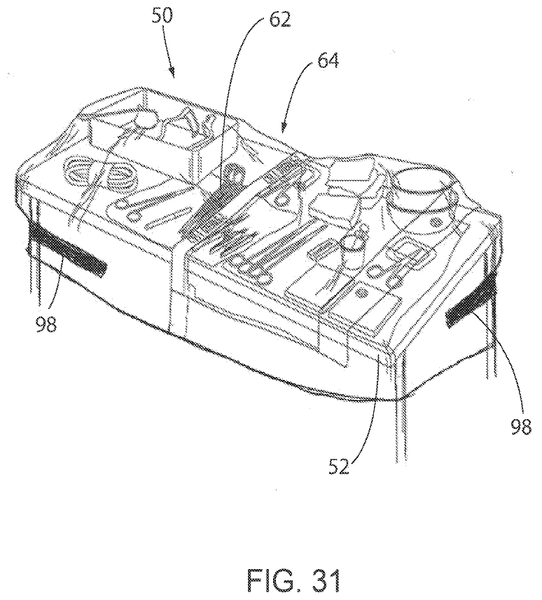

Alternatively, a poly-tie 98 or a plurality of poly-ties or pull tabs could be used to secure the drape 64 about the table 52. For instance, in FIG. 9, there are poly-ties 98 located adjacent to each corner of the table 52. Each poly-tie 98 has a first end 100, a second end 102, and a body 101 located therebetween. The first end 100 is permanently affixed to the drape 64, and the second end 102 has an adhesive 104 located thereon with a release liner 106 that can be peeled off to expose the adhesive 104. The second end 102 is then pulled around the corner such that the poly-tie 98 fits tightly around the corner, and the adhesive 104 is applied to the drape 64. Once in position, the first side 84 of the adhesive sticker 82 is applied to the second end 102 of the poly-tie 98, and the second side 86 of the adhesive sticker 82 is applied to the drape 64. In the event that the second side 86 is disengaged from the drape 64, the adhesive sticker 82 will be broken about the perforation 92. As a result, it will be easy for an observer to locate the broken adhesive sticker 82, which would indicate that a breach of sterility is possible. A similar embodiment is shown in FIG. 31, where poly-ties or pull tabs 98 are located at opposite corners of the drape 64, which can be pulled in opposite directions to gather material of the drape 64 in a snug manner. Further still, poly-ties or pull tabs 98 may be located at every corner of the drape 64 to allow for securement of the drape 64 to the table 52 at every corner.

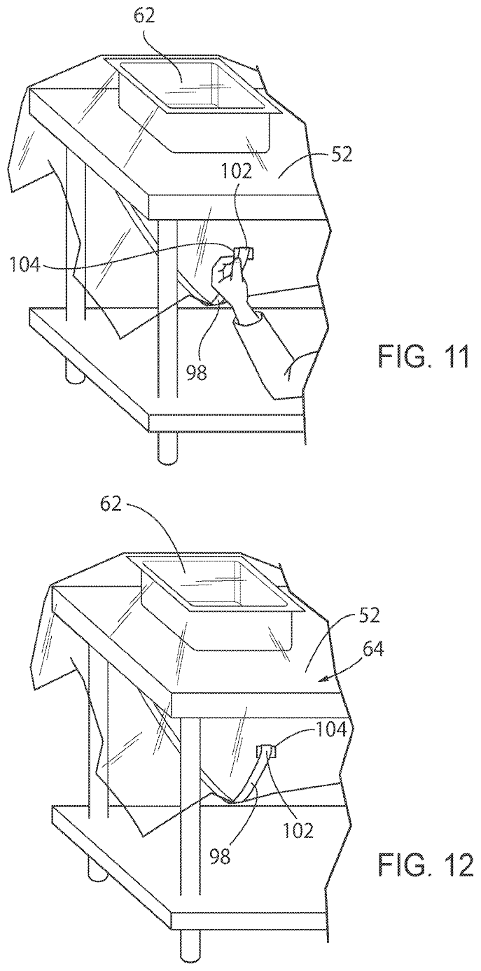

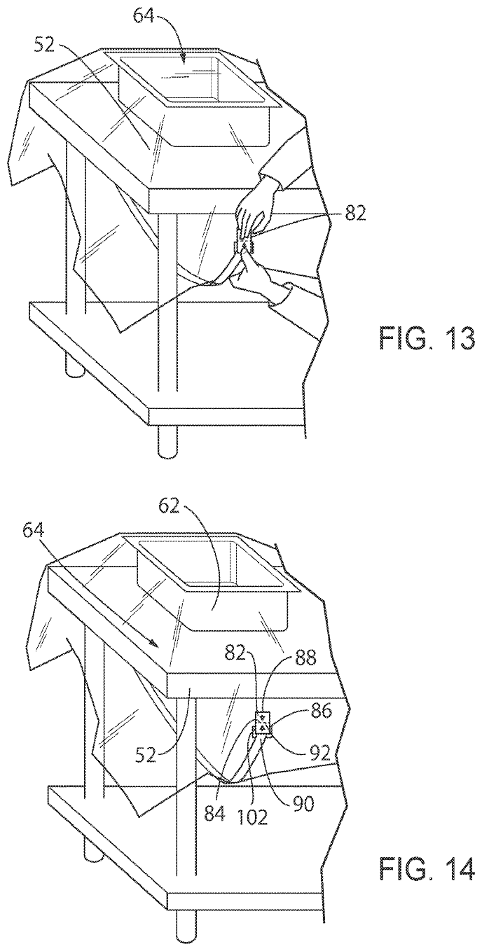

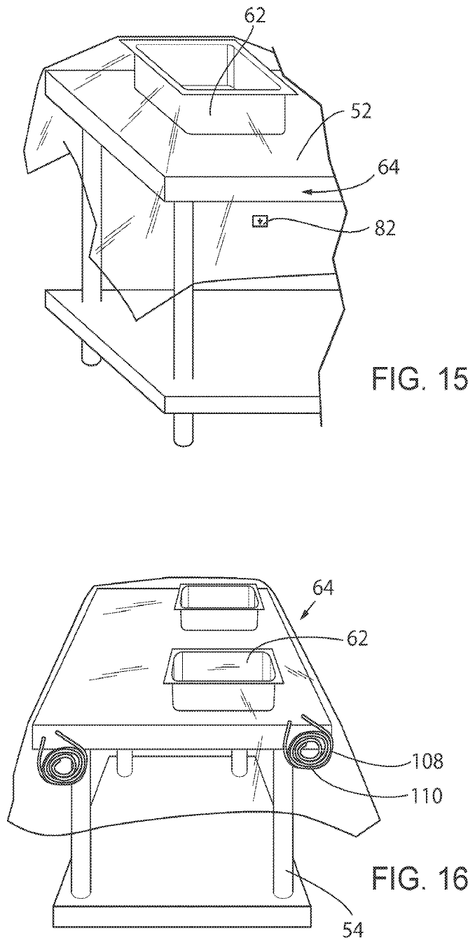

Turning next to FIG. 10, another embodiment of a drape system 50 with a poly-tie 98 or a plurality of poly-ties is shown. In this embodiment, the poly-tie 98 is fed under the underside 56 of the table 52. As shown in FIG. 10, the release liner 106 is removed, and the adhesive 104 is affixed to the side of the sterile drape 64, as shown in FIGS. 11 and 12. Alternatively, the poly-tie 98 can be fed through a buckle (not shown) to secure the poly-tie 98 and the drape 64 about the table 52. Next, the adhesive sticker 82 is applied, with the first side 84 being applied to the second end 102 of the poly-tie 98, and the second side 86 of the sticker 82 being applied to the drape 64, as shown in FIGS. 13 and 14, or a part of the table 52 or legs 54. Once the second end 102 of the poly-tie 98 is detached from the drape 64, the adhesive sticker 82 will break about the perforation 92, as shown in FIG. 15. When this occurs, an observer can easily determine that a breach of sterility is possible.

Additionally, a single poly-tie 98 can be used to wrap around the entire perimeter 60 of the table 52. In such an embodiment, loops or hooks (not shown) can be included around the drape 64 or the perimeter 60 of the table 62. The poly-tie 98 can be guided into these loops or hooks so that it is easier to incrementally wrap the poly-tie 98 around the entire perimeter 60 of the table 62. The adhesive sticker 82 can then be applied as described above.

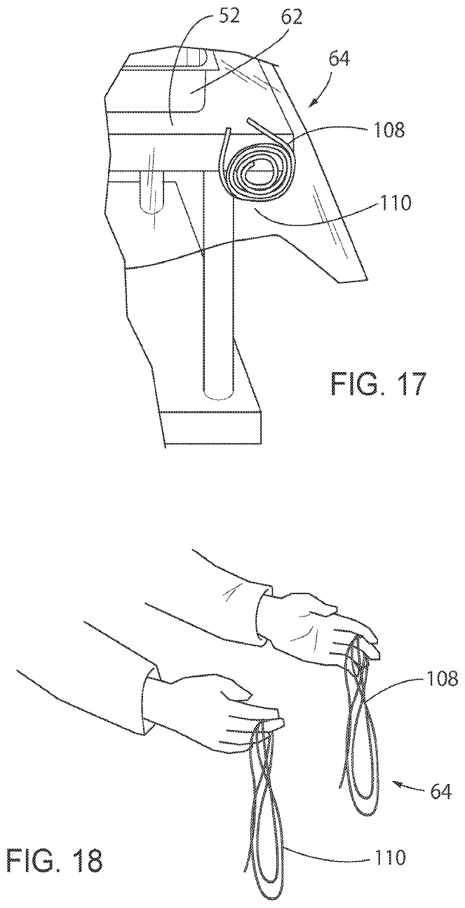

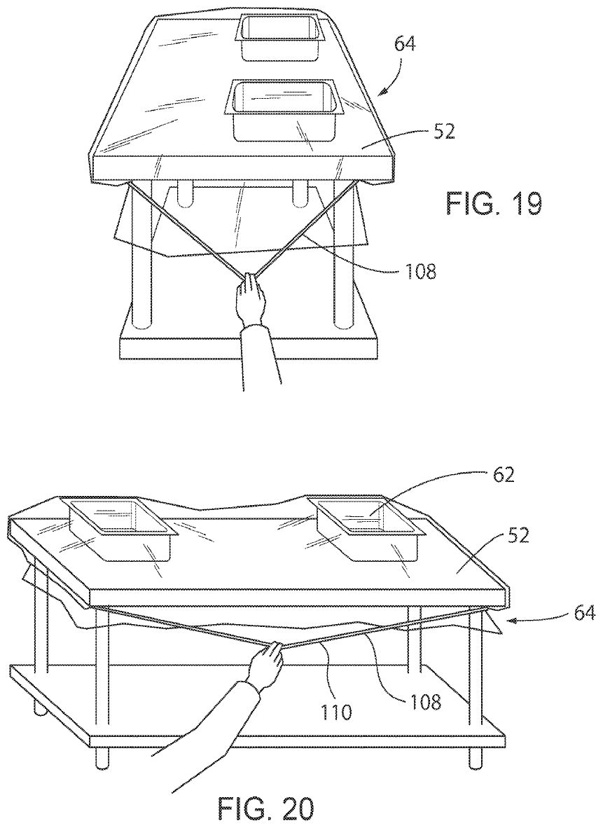

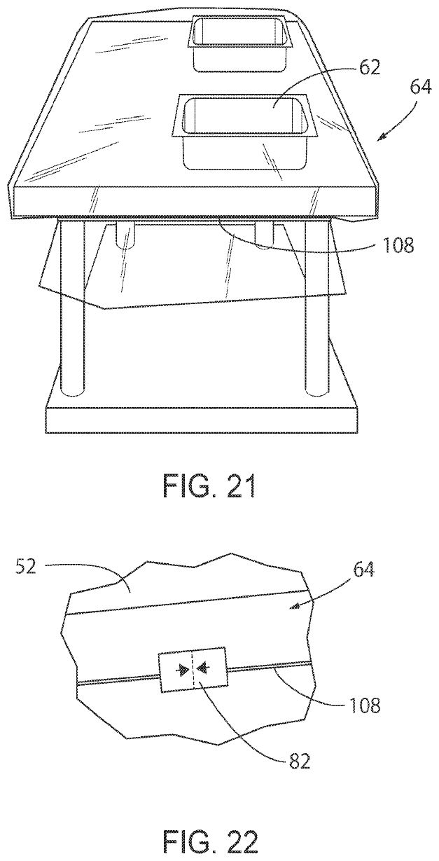

In yet another embodiment shown in FIGS. 16-22, the drape 64 is secured to the table 52 using a plurality of rubber bands 108 and hooks 110. The rubber bands 108 and hooks 110 may come wrapped within the drape 64, as shown in FIGS. 16 and 17. The rubber bands 108 and hooks 110 are removed from the drape 64, as shown in FIG. 18. Each hook 110 is then used to engage two adjacent rubber bands 108. These rubber bands 108 and hooks 110 wrap around the table 52 beneath the perimeter 60 to secure the drape 64 to the table 52. In FIGS. 19 and 20, the rubber bands 108 and hooks 110 are pulled away from the perimeter 60 so that they can more easily be seen. Once the rubber bands 108 and hooks 110 are appropriately located, as shown in FIG. 21, the first side 84 of the adhesive sticker 82 is attached to the hooks 110, and the second side 86 of the adhesive sticker 82 is attached to the drape 64 as shown in FIG. 22. In this way, when the hooks 110 are disengaged from the rubber bands 108, the adhesive sticker 82 will break about the perforation 92, which makes it easy to visualize when a sterility breach may have occurred.

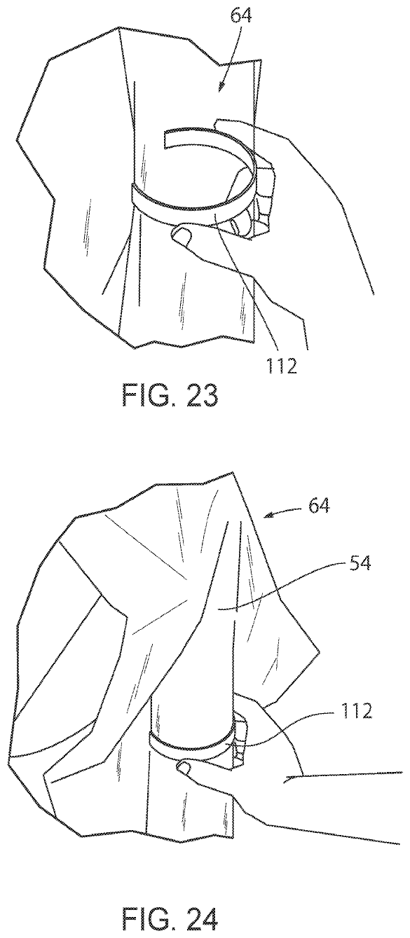

Looking now to FIGS. 23-24, the drape 64 can be secured to the table 52 using clips 112 that are configured to be releasably secured to the legs 54 of the table 52. The clips 112 could be made of any suitable material, including for instance plastic and metal. In this embodiment, the drape 64 is placed upon the table 52 such that the first edge 66, second edge 68, first end 70, and second end 72 fall down around the table 52 adjacent to the legs 54. The drape 64 is then pulled up against the legs 54. Clips 112 are installed that hold the drape 64 tightly against the legs 54. Next, the first side 84 of the adhesive sticker 82 is attached to the clip 112, and the second side 86 of the adhesive sticker 82 is attached to the drape 64 or a portion of the table 52. In the event that the clip 112 is removed, the adhesive sticker 82 will break about the perforation 92 so that it is easy to visually determine whether potential breaches of sterility have occurred. Alternatively, the clip 112 may be configured to break, once removed from the legs 54. In this embodiment, the broken clip 112 will serve as evidence that a potential breach of sterility has occurred.

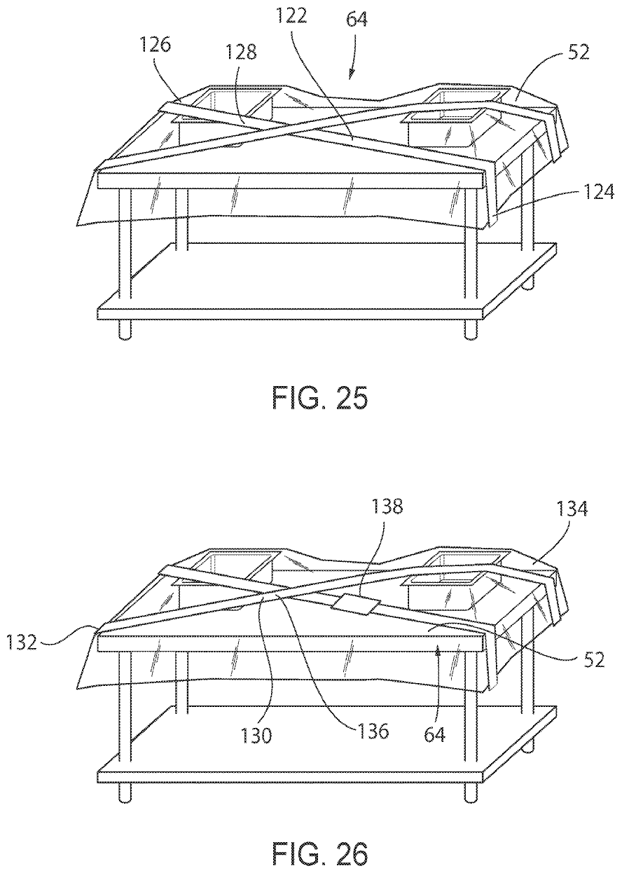

FIG. 25 illustrates another embodiment with a strap 122 that is used to secure the drape 64 about the table 52. The strap 122 can have a first end 124 that attaches to one leg, a second end 126 that attaches to another leg, and a body 128 extending from the first end 124 to the second end 126. Multiple straps could be used, for instance, as shown forming an “X” shape or a “Z” shape (not shown). Alternatively, the strap 122 could simply be a strip of tape 130, as seen in FIG. 26. The strip of tape 130 could have a first end 132 that attaches to one leg, a second end 134 attached to another leg, and a body 136 in between that secures the drape 64 to the table 52. In either case, the strap 122 or tape 130 secures the drape 64 to the table 52. The adhesive sticker 82 is then applied to one of the ends and the drape 64 or part of the table 52. If the end is moved away from the drape 64 or table 52, the adhesive sticker 82 will break about the perforation 92 so that it is easy to visually determine whether potential breaches of sterility have occurred.

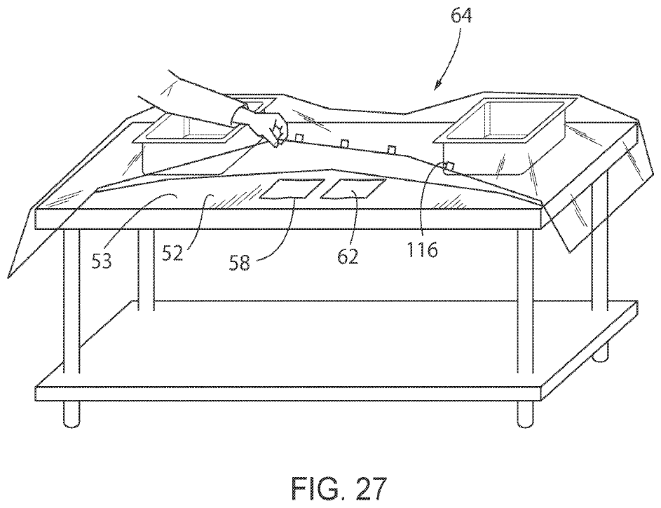

Additionally, turning to FIG. 27 a plurality of adhesive tabs 116 could be installed on an underside of the drape 64, each of which are covered by release liners (not shown). As the drape 64 is unfolded, the release liners are removed to expose the adhesive tabs 116. The tabs 116 are then pressed against the top surface of the table 52 or the table cover to secure the drape 64 to the table 52. In the event that any of the adhesive tabs 116 are separated from the top surface of the table 52 or the table cover 53, an observer will easily be able to see this and know that a potential breach of sterility has occurred. Similarly, there could be perforations (not shown) around each tab 116. Again, an observer could easily view the tabs 116 to see if any perforations have been broken, which could mean that sterility has been compromised. Additionally, a perimeter perforation (not shown) may be formed around the first edge 66, second edge 68, first end 70, and second end 72 of the drape 64.

Any of the drape systems 50 described above may include a time stamp 138 as shown in FIG. 26. The time stamp 138 may include any information relevant to the preparation of the drape system 50 about the table 52, including the time the drape system 50 was prepared, identification of the individual who prepared the drape system 50, information describing the specific medical procedure that the drape system 50 was prepared for, when the medical procedure is scheduled to occur, information relating to the patient, and any other information that could be useful to an individual who eventually uses the drape system 50. The time stamp 138 could be handwritten, it could be automatically printed from a printer and applied to the drape 64, or an RF/ID tag could be utilized, which would allow a smartphone, tablet, scanner, etc. to scan the table and capture date and time information once the drape 64 is placed.

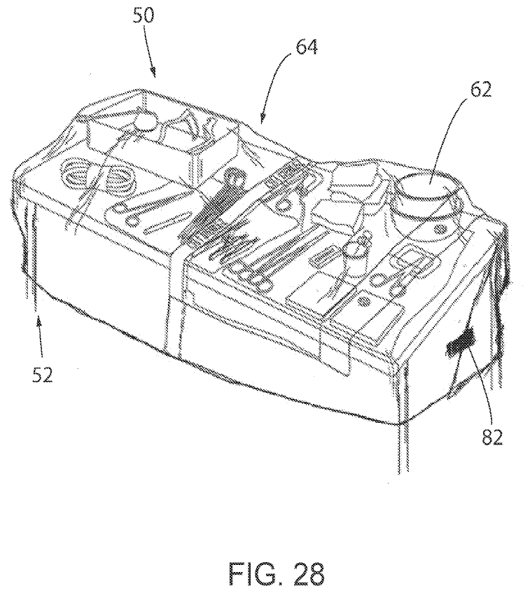

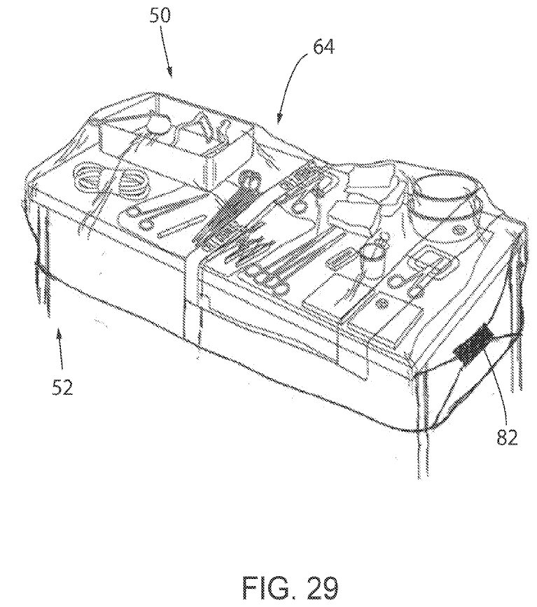

Turning next to FIG. 28, another embodiment of the drape system 50 is provided. In this embodiment, the sterile drape 64 is a flat sheet dimensioned to have excess material relative to the dimensions of the surgical table 52. This excess material can be folded back onto itself to result in an overlapping of material. Once the material has been folded back onto itself, an adhesive sticker 82 may be applied to gather the material together. As a result, the sterile drape 64 is secured relative to the surgical table 52 and forms a snug fit of the drape 64 relative to the table 52. As shown, the drape 64 is gathered at either end of the table 52, although it should be noted that the drape 64 could similarly be gathered on either side of the table 52. Somewhat similarly, looking to FIG. 29, the sterile drape 64 is a flat sheet hat may be folded onto itself twice so that the drape 64 is snugly fitted relative to the table 52. Of course, the drape 64 could similarly be folded additional times in order to ensure a snug fitting relative to the table 52. Again, an adhesive sticker 82 may be added to hold the gathered material together and to secure the drape 64 in a secure manner. This forms a snug fit of the drape 64 relative to the table 52.

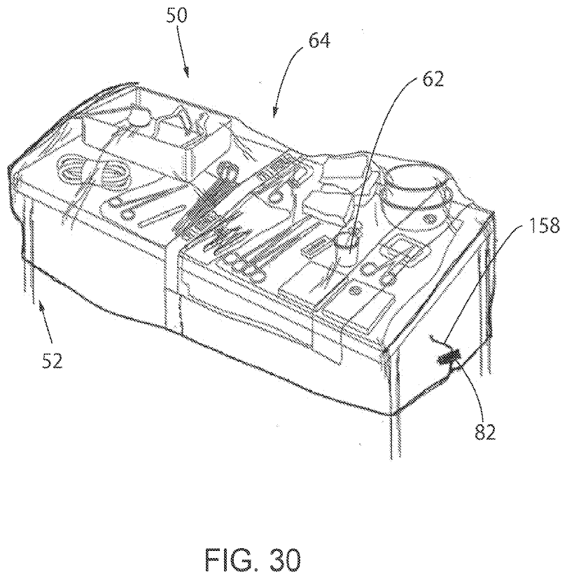

FIG. 30 shows yet another embodiment of the drape system 50. More specifically, the sterile drape 64 has a slit 158 formed therein. For instance, as shown the slit 158 is formed in one end of the drape 64. Slits 158 could similarly be formed in both ends of the drape 64 of the sides of the drape 64. Material on either side of the slit 158 can be gathered to overlap, and then an adhesive sticker 82 can be applied to either side of the slit. As a result, the drape 64 is snugly secured to the surgical table 52.

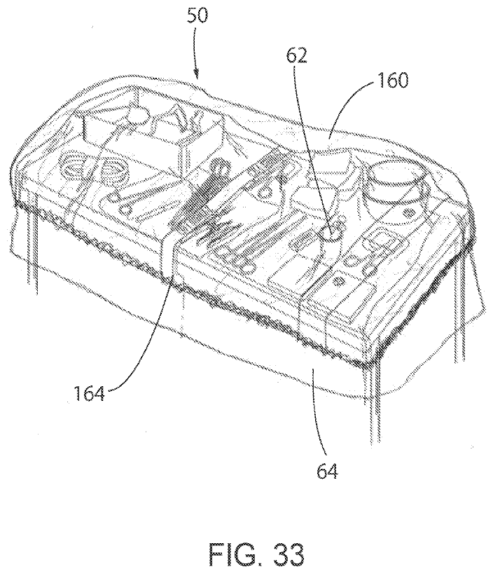

Further still, FIG. 33 shows another embodiment of the drape system 50 that includes the sterile drape 64 that is a flat sheet, as well as an additional dome bag 160. The dome bag 160 includes a sheet 162 with a gathering component 164 located adjacent to the perimeter of the sheet. For instance, the gathering component 164 could be an elastic band or other material capable of securing the dome bag 160 relative to the drape 64 and table 52. The dome bag 160 is configured to be placed over the sterile drape 64.

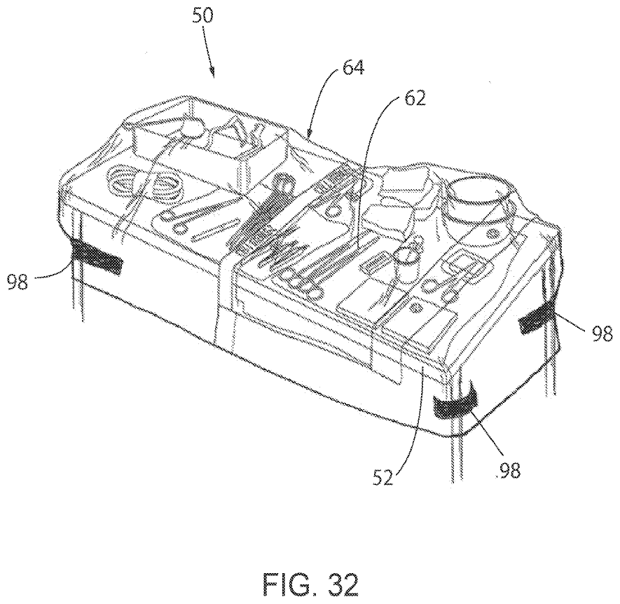

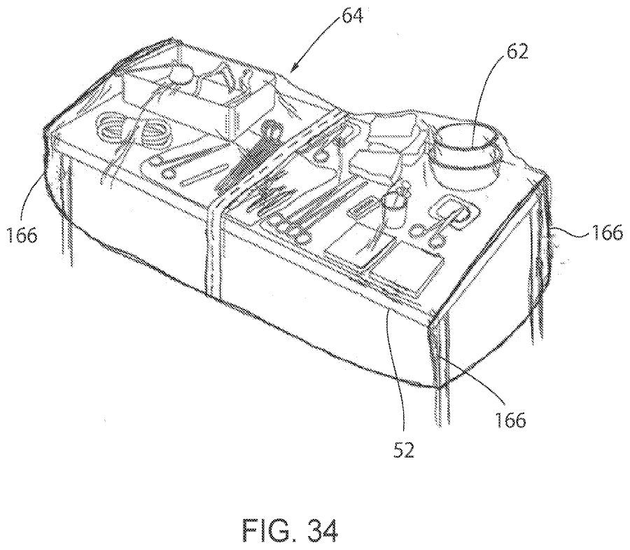

Additionally, FIG. 34 shows a drape system 50 having a drape 64 with vertical seals 166 located at the corners of the drape 64. The vertical seals 166 may be configured to securely fit around the table. Stated differently, the seals 166 may be located at a distance from one another that is substantially the same distance as the corners of the table 52. This would ensure that the drape 64 fits snugly relative to the table. Drapes 64 having various dimensions may be provided depending on the desired size of the table. This would reduce the excess material of the drape 64 present in some of the other embodiments described above. For instance, such a drape 64 could be used in substation with the flat drape 64 shown in FIGS. 30-32.

Additionally, a method of maintaining sterility about a surgical back table 52 is provided. This method includes initially placing a sterile cover 53 on a top surface 58 of the back table 52. A plurality of sterile surgical instruments 62 is then placed on the sterile cover 53. Once the surgical instruments 62 are appropriately located, a sterile drape 64 is placed on top of the sterile surgical instruments 62 and the sterile cover 53. A securement device 80 is then used to secure the sterile drape 64 to the surgical table 52. The securement device 80 is configured to indicate when the drape 64 has been removed from the surgical table 52 to ensure sterility is maintained about the top surface 58 and the plurality of surgical instruments 62. Also, an adhesive sticker 82 can be provided as the securement device 80, where the adhesive sticker 82 has a first side 84, a second side 86, a third side 88, a fourth side 90, and a perforation 92 that extends between the first side 84 and the second side 86. The third side 88 of the sticker 82 is then attached to one of a plurality of legs 54 supporting the table 52 and the fourth side 90 of the sticker 82 is attached to the sterile drape 64. In the event that the drape 64 is moved, the adhesive sticker 82 will break in half about the perforation 92 when the third side 88 is pulled away from the fourth side 90 of the adhesive sticker 82.

All the disclosed embodiments are useful in conjunction with medical operations where breaches of sterility are dangerous and costly. There are virtually innumerable uses for the present invention, all of which need not be detailed here. All the disclosed embodiments can be practiced without undue experimentation.

Although the best mode contemplated by the inventors of carrying out the present invention is disclosed above, practice of the present invention is not limited thereto. It will be manifest that various additions, modifications, and rearrangements of the features of the present invention may be made without deviating from the spirit and scope of the underlying inventive concept.

For example, any of the drape systems disclosed in U.S. Pat. No. 8,726,907 and Application Publication Nos. 2014/0251346 and 2015/0374442, which are incorporated herein by reference in full, could be used with the drape system 50 and securement device 80 described herein. Similarly, any material could be used as long as sterility is maintained. In this way, the drape 64 and securement device 80 could be fabricated from virtually any suitable materials.

Moreover, the individual components need not be formed in the disclosed shapes, or assembled in the disclosed configuration, but could be provided in virtually any shape and assembled in virtually any configuration. Further, although the drape 64 and securement device 80 described herein are physically separate modules, it will be manifest that they may be integrated into the apparatus with which it is associated. Furthermore, all the disclosed features of each disclosed embodiment can be combined with, or substituted for, the disclosed features of every other disclosed embodiment except where such features are mutually exclusive.

It is intended that the appended claims cover all such additions, modifications, and rearrangements. Expedient embodiments of the present invention are differentiated by the appended claims.

What is claimed is:

1. A drape system for use with a surgical table supported by a plurality of legs with an underside and a top surface with a perimeter, where the top surface supports a plurality of surgical instruments comprising:
a sterile drape comprising:
an area defined by a predetermined length and a predetermined width;
a selectively separable portion extending a substantial portion of at least one of the predetermined length and predetermined width;
the selectively separable portion further comprising a first peripheral edge and a second peripheral edge, which are normally in a sealed position and adapted to be unsealed by applying opposing forces to the first and second peripheral edges; and
the selectively separable portion further comprising at least a first overlaying section, which overlays the selectively separable portion when the sterile drape is in a sealed position;
at least one securement device for securing the sterile drape to the surgical table, wherein the securement device is configured to be attached to the surgical table and indicate when the sterile drape has been removed from the surgical table to ensure sterility is maintained about the top surface and the plurality of surgical instruments, wherein the securement device comprises an adhesive sticker with a first side, a second side opposite the first side, a third side extending between the first side and the second side, a fourth side extending between the first side and the second side and located opposite the third side, and a perforation extending from the first side to the second side, where the perforation is configured to break when the adhesive sticker is pulled apart, wherein the third side of the adhesive sticker is attached to the sterile drape, and the fourth side is configured to be attached to one of the underside or one of the plurality of legs.

2. The drape system of claim 1, further comprising:
a plurality of rubber bands; and
a plurality of hooks used to engage adjacent rubber bands;
wherein the plurality of rubber bands and the plurality of hooks are configured to wrap around the perimeter; and
wherein the third side of the adhesive sticker is attached to one of the hooks, and the fourth side of the adhesive sticker is attached to the sterile drape.

3. A method of maintaining sterility about a back table comprising the steps of:
placing a sterile cover on a top surface of the back table;
placing a plurality of sterile surgical instruments on the sterile cover;
placing a sterile drape on top of the sterile surgical instruments; and
attaching a securement device of the sterile drape to the surgical table to secure the sterile drape to the surgical table, where the securement device is configured to indicate when the drape has been removed from the surgical table to ensure sterility is maintained about the top surface and the plurality of surgical instruments, wherein the securement device comprises an adhesive sticker having a first side, a second side, a third side, a fourth side, and a perforation located between the third side and the fourth side, and the attaching step comprises:
attaching the third side of the adhesive sticker to one of a plurality of legs supporting the table; and
attaching the fourth side of the adhesive sticker to the sterile drape; and
breaking the adhesive sticker in half about the perforation by pulling the third side of the adhesive sticker away from the fourth side of the adhesive sticker.

4. The drape system of claim 1, further comprising one of a magnet and a weight that is rested on a top of the sterile drape to secure the sterile drape to the top surface of the surgical table.

5. The drape system of claim 1, further comprising at least one poly-tie with each poly-tie having:
a first end permanently attached to the sterile drape;
a second end with an adhesive; and
a release liner covering the adhesive;
wherein the release liner is removed to expose the adhesive;
wherein the second end is pulled away from the first end; and
wherein the adhesive is secured to the sterile drape to secure the sterile drape to the table.

6. The drape system of claim 5, wherein the third side of the adhesive sticker is attached to the second end of the poly-tie, and the fourth side of the adhesive sticker is attached to the sterile drape.

7. The drape system of claim 1, further comprising: a plurality of clips that releasably attach to the plurality of legs, where the clips are configured to secure the sterile drape to the plurality of legs;
wherein the third side of the adhesive sticker is attached to the clip, and the fourth side of the adhesive sticker is attached to the sterile drape.

8. The drape system of claim 1, further comprising:
a poly-tie attached to the first peripheral edge; and
a buckle attached to the second peripheral edge;
wherein the poly-tie is passed beneath the underside and is secured within the buckle; and
wherein the third side of the adhesive sticker is attached to the poly-tie, and the fourth side of the adhesive sticker is attached to the sterile drape.

9. The drape system of claim 1, wherein the securement device further comprises:
a plurality of adhesive tabs located on a bottom side of the sterile drape; and
a plurality of release liners releasably attached to the adhesive tabs;
wherein the plurality of release liners is removed to expose the plurality of adhesive tabs; and
wherein the plurality of adhesive tabs is configured to be anchored to the top surface.

10. The drape system of claim 1, wherein the securement device further comprises:
a plurality of adhesive tabs located on a bottom side of the sterile drape;
a plurality of release liners releasably attached to the adhesive tabs; and
a second perforation extending around the sterile drape;
wherein the plurality of release liners is removed to expose the plurality of adhesive tabs; and
wherein the plurality of adhesive tabs is configured to be anchored to the perimeter.

11. The drape system of claim 1, wherein the securement device further comprises:
at least one strap with a first end, a second end, and a body located therebetween, where the first end is configured to be attached to one of the plurality of legs, the body secures the sterile drape to the table, and the second end is attached to another one of the plurality of legs;
wherein the third side of the adhesive sticker is attached to one of the first end and the second end, and the fourth side of the adhesive sticker is configured to be attached to one of the plurality of legs.

12. The drape system of claim 11, wherein the at least one strap is reusable.

13. The drape system of claim 1, wherein the securement device further comprises:
at least one strip of tape with a first end, a second end, and a body therebetween, where the first end of the tape is configured to be attached to one of the plurality of legs, the body is secured to the sterile drape, and the second end of the tape is configured to be attached to one of the plurality of legs.

14. The drape system of claim 1, further comprising a time stamp indicating when the sterile drape is secured to the surgical table.
